(19) United States
(12) Patent Application Publication
(10) Pub. No.: US 2022/0192583 A1
(43) Pub. Date: Jun. 23, 2022

(54) DETECTING SENTINEL LYMPH NODES DURING SURGERY

(71) Applicants: Mohammad Abdolahad, Tehran (IR); Ashkan Zandi, Tehran (IR); Amir Mamdouh, Tehran (IR); Alireza Madannejad, Tehran (IR); Yasin Kordehlachin, Tehran (IR); Seyed Mohammad Sadegh Mousavi-Kiasary, Tehran (IR); Fereshteh Abbasvandi, Tehran (IR)

(72) Inventors: Mohammad Abdolahad, Tehran (IR); Ashkan Zandi, Tehran (IR); Amir Mamdouh, Tehran (IR); Alireza Madannejad, Tehran (IR); Yasin Kordehlachin, Tehran (IR); Seyed Mohammad Sadegh Mousavi-Kiasary, Tehran (IR); Fereshteh Abbasvandi, Tehran (IR)

(73) Assignee: Nano Hesgarsazan Salamat Arya, Tehran (IR)

(21) Appl. No.: 17/568,335

(22) Filed: Jan. 4, 2022

Related U.S. Application Data
(60) Provisional application No. 63/133,432, filed on Jan. 4, 2021.

(51) Int. Cl. A61B 5/00; A61B 5/0507; C12Q 1/6886

(52) U.S. Cl. CPC A61B 5/418; C12Q 1/6886; A61B 5/0507

(57) ABSTRACT

A method for detecting sentinel lymph nodes during surgery. The method includes injecting a crystalloid solution into a region, measuring a plurality of electrical parameters utilizing a sensor, and detecting a sentinel lymph node among a plurality of lymph nodes based on the plurality of electrical parameters. The region is associated with a tumor in a patient's body. Measuring the plurality of electrical parameters includes measuring each respective electrical parameter of a respective lymph node of the plurality of lymph nodes. The plurality of lymph nodes are associated with the tumor. The sentinel lymph node is located at a shortest distance from the tumor among the plurality of lymph nodes.

### CROSS-REFERENCE TO RELATED APPLICATION

This application claims the benefit of priority from pending U.S. Provisional Patent Application Ser. No. 63/133,432, filed on Jan. 4, 2021, and entitled “SYSTEM FOR DETECTING CANCEROUS LYMPH NODES,” which is incorporated herein by reference in its entirety.

### TECHNICAL FIELD

The present disclosure generally relates to medical diagnosis, and particularly, to cancer diagnosis and treatment.

### BACKGROUND

Early surgical removal of cancerous tissues, as well as lymph nodes in areas surrounding cancerous tissues is generally carried out to completely arrest cancer progress. However, during a surgical removal of a cancerous tissue, it may be not known if cancer has spread to lymph nodes or not. Therefore, a surgeon may unnecessarily perform a complete lymph node dissection. This procedure may be painful and may impose a heavy burden on patients.

Cancer is not randomly transferred to lymph nodes, but the spread of cancer may follow an orderly pattern, in which cancer may first spread to regional lymph nodes close to a tumor and then, due to directional nature of the flow of a lymph, may spread to other lymph nodes in a predictable fashion. A first draining lymph node or group of lymph nodes may be referred to as sentinel lymph nodes. If a sentinel lymph node corresponding to a primary cancer lesion site is located and found not to have been involved in cancer, a total lymph node dissection may be avoided.

Accordingly, locating a sentinel lymph node before tissue removing surgery may be important. One way to detect a sentinel lymph node is to use a blue colorant method, in which a blue colorant may be injected into a tumor about 20 minutes before tumor removal surgery. The blue colorant may move toward lymph nodes and may reach a sentinel lymph node within 5 to 15 minutes after injection and the surgeon may visually detect the sentinel lymph node. However, fatty tissue surrounding lymph nodes may have to be first peeled away so that visual detection may be possible. This may be time-consuming and an injected blue colorant may have enough time to move forward beyond a sentinel lymph node and reach other lymph nodes and render visual detection of sentinel lymph nodes impossible.

Another method for detecting sentinel lymph nodes is to utilize radioactive materials, radioisotropic materials, or radiopharmaceuticals. In this approach, a radiopharmaceutical such as Technetium 99 m, Indium 111, Iodine 123, or Iodine 125 may be preoperatively injected into a tumor and a gamma probe may be utilized for intraoperative detection of a sentinel lymph node by detecting a gamma radiation emitted by the sentinel lymph node. However, managing of surgical procedure during radiation may be complicated and also utilization of a gamma probe may be time-consuming. As a result, an injected radiopharmaceutical may reach lymph nodes beyond the sentinel lymph node in a short time or an amount of gamma radiation may drop below a detectable level due to the complexity of preparing a patient for surgery. Furthermore, this approach may have a high risk for a patient's health due to utilizing radioactive radiation.

There is, therefore, a need for a method that may provide easier and more accurate intraoperative detection of sentinel lymph nodes while performing sentinel node navigation surgery without a need for injection of hazardous radioactive materials. There is also a need for a system that may be easily utilized for locating sentinel lymph nodes which may allow for avoiding unnecessary lymph node dissection.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for detecting sentinel lymph nodes during surgery. An exemplary method may include injecting a crystalloid solution into a region, measuring a plurality of electrical parameters utilizing a sensor, and detecting a sentinel lymph node among a plurality of lymph nodes based on the plurality of electrical parameters utilizing one or more processors. An exemplary region may be associated with a tumor in a patient's body. In an exemplary embodiment, measuring the plurality of electrical parameters may include measuring each respective electrical parameter of a respective lymph node of the plurality of lymph nodes. Exemplary plurality of lymph nodes may be associated with the tumor. An exemplary sentinel lymph node may be located at a shortest distance from the tumor among the plurality of lymph nodes.

In an exemplary embodiment, injecting the crystalloid solution may include injecting one of a normal saline, a dextrose solution, or a Ringer's solution into the region. In an exemplary embodiment, injecting the crystalloid solution into the region may include injecting the crystalloid solution into the tumor.

In an exemplary embodiment, utilizing the sensor may include inserting a probe of a flexible gigahertz antenna into each of the plurality of lymph nodes, measuring each of the plurality of electrical parameters by the probe, transmitting each of the plurality of electrical parameters from the probe to a signal transmission unit via a detachable cable, and transmitting the plurality of electrical parameters to the one or more processors from the signal transmission unit. An exemplary flexible gigahertz antenna may include a flexible coplanar waveguide (CPW) antenna. In an exemplary embodiment, inserting the probe into each of the plurality of lymph nodes may include wearing a glove by an operator of the sensor, placing a mounting member on one of a fingers of the glove, and attaching the probe onto the mounting member.

In an exemplary embodiment, detecting the sentinel lymph node may include finding a largest electrical parameter of the plurality of electrical parameters and identifying a lymph node of the plurality of lymph nodes that may include the largest electrical parameter as the sentinel lymph node. An exemplary largest electrical parameter may include a highest magnitude among the plurality of electrical parameters.

In an exemplary embodiment, finding the largest electrical parameter may include measuring respective variations of each of the plurality of electrical parameters in a frequency domain utilizing the sensor, obtaining a plurality of maximum values by finding each respective maximum value of respective variations of each of the plurality of electrical parameters in a predetermined frequency range of the frequency domain, and obtaining the largest electrical parameter by finding a largest maximum value among the plurality of maximum values. In an exemplary embodiment, measuring respective variations of each of the plurality of electrical parameters may include measuring respective variations of one of a plurality of scattering parameters, a plurality of electrical conductivities, or a plurality of relative permittivities in the frequency domain for each respective lymph node of the plurality of lymph nodes.

In an exemplary embodiment, finding each respective maximum value of respective variations of each of the plurality of electrical parameters in the predetermined frequency range may include finding each respective maximum value of respective variations of each of the plurality of relative permittivities for each respective lymph node of the plurality of lymph nodes in a frequency range of 2 GHz to 4 GHz in the frequency domain.

An exemplary method may further include detecting metastatic cancer cells inside the sentinel lymph node by inserting a needle of the sensor into the sentinel lymph node, measuring one of an aqueous carbon dioxide (CO₂) concentration or a pH concentration of the sentinel lymph node utilizing the needle, and identifying the metastatic cancer cells inside the sentinel lymph node based on the one of the aqueous CO₂ concentration or the pH concentration. An exemplary the needle may include one of a miniaturized CO₂ sensor needle or a miniaturized pH sensor needle.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for detecting sentinel lymph nodes during surgery, consistent with one or more exemplary embodiments of the present disclosure.

FIG. 1B shows a flowchart for detecting a sentinel lymph node, consistent with one or more exemplary embodiments of the present disclosure.

FIG. 1C shows a flowchart for finding a largest electrical parameter, consistent with one or more exemplary embodiments of the present disclosure.

FIG. 1D shows a flowchart for detecting metastatic cancer cells inside a lymph node, consistent with one or more exemplary embodiments of the present disclosure.

FIG. 2 shows a schematic of a system for o detecting sentinel lymph nodes during surgery, consistent with one or more exemplary embodiments of the present disclosure.

FIG. 3 shows a schematic of utilizing a system for detecting sentinel lymph nodes during surgery, consistent with one or more exemplary embodiments of the present disclosure.

FIG. 4A shows a schematic of a flexible coplanar waveguide (CPW) antenna, consistent with one or more exemplary embodiments of the present disclosure.

FIG. 4B shows a schematic of a flexible CPW antenna with a slot, consistent with one or more exemplary embodiments of the present disclosure.

FIG. 5A shows measured variations of an S-parameter in a lymph node, consistent with one or more exemplary embodiments of the present disclosure.

FIG. 5B shows measured variations of a relative permittivity in an axillary lymph node before and after injecting a dextrose solution, consistent with one or more exemplary embodiments of the present disclosure.

FIG. 5C shows measured variations of a relative permittivity in an axillary lymph node before and after injecting a Ringer's solution, consistent with one or more exemplary embodiments of the present disclosure.

FIG. 5D shows measured variations of a relative permittivity in an axillary lymph node before and after injecting a normal saline solution, consistent with one or more exemplary embodiments of the present disclosure.

FIG. 6 shows an example computer system in which an embodiment of the present invention, or portions thereof, may be implemented as computer-readable code, consistent with exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for locating sentinel lymph nodes associated with primary cancer lesion sites during surgery, for example, during a breast cancer surgery. An exemplary sentinel lymph node may refer to a nearest lymph node to an exemplary tumor. Therefore, if an exemplary solution is injected into the tumor, a larger amount of the solution may be eventually received by the sentinel lymph node than other lymph nodes. As a result, if an electrically conductive solution is injected into the tumor, electrical properties of an exemplary sentinel lymph node may experience a larger change than other lymph nodes due to presence of a larger amount of injected solution in the sentinel lymph node. Therefore, an exemplary sentinel lymph node may be detected by finding a lymph node with a more considerable change in its electrical properties.

Based on the above, an exemplary method for locating sentinel lymph nodes may be designed and implemented. An exemplary method may include injecting a crystalloid solution (such as saline, which is electrically conductive) into a tumor. Next, an exemplary method may measure changes of electrical properties (such as relative permittivity) of lymph nodes around the tumor caused by a discharge of the injected crystalloid solution into regional lymph nodes. An exemplary method may identify a lymph node with a largest change of its electrical properties as the sentinel lymph node.

An exemplary method may utilize a sensor for measuring electrical properties of lymph nodes around a tumor. An exemplary gigahertz antenna may be used as a sensor to achieve an accurate, low-risk and high-reliability diagnosis for detection of sentinel lymph nodes. An exemplary antenna may operate in a wide frequency range and a flexible poly ethylene terephthalate (PET) substrate may be used in design of the antenna. Due to the use of a flexible substrate, a surgeon's maneuverability while touching lymph nodes may be improved and the antenna dimensions may be reduced to make it easier to use the antenna during surgery by a surgeon. By placing an exemplary antenna on all the lymph nodes and comparing their electrical parameters, a lymph node with larger change in electrical properties (for example, a higher relative permittivity) may be identified as a sentinel lymph node.

Utilizing crystalloid solutions may allow for a simple, safe, and accurate detection of sentinel lymph nodes. Since exemplary crystalloid solutions are not harmful to a patient's body, in cases where an amount of an exemplary crystalloid solution within lymph nodes drops below a detectable level, more solution may be injected into a tumor's vicinity to facilitate associated measurements.

An exemplary method may further detect cancerous lymph nodes during surgery. In an exemplary embodiment, metastatic tumor cells within lymph nodes may undergo a metabolic shift toward fatty acid oxidation (FAO) and transcriptional coactivator yes-associated protein (YAP) may be selectively activated in such metastatic tumors. Carbon dioxide (CO₂) and water may be two products of FAO, which may lead to formation of carbonic acid (H₂CO₃) within a cancerous lymph node. An exemplary method may intraoperatively measure concentration of H₂CO₃ or aqueous CO₂ within a sentinel lymph node and may identify the sentinel lymph node as cancerous if a concentration of H₂CO₃ or aqueous CO₂ is higher than a predetermined threshold. Since FAO creates an acidic environment within a lymph node, an exemplary method may also measure pH of the lymph node in order to determine if the lymph node is cancerous or not. An exemplary sensor may be inserted within a detected sentinel lymph node and based on concentration and/or pH measurements, lymph node metastasis may be detected. Such configuration of an exemplary sensor may allow for detecting lymph node metastasis without a need for removing the lymph nodes from a patient's body, which may improve the patient's quality of life.

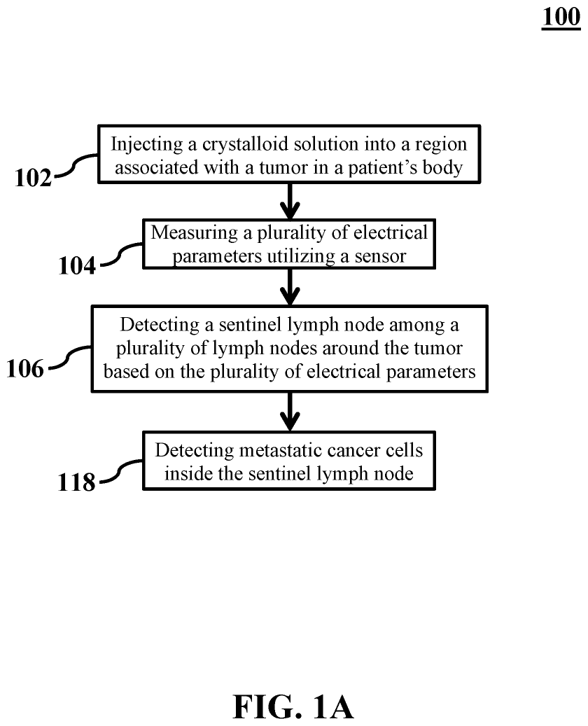

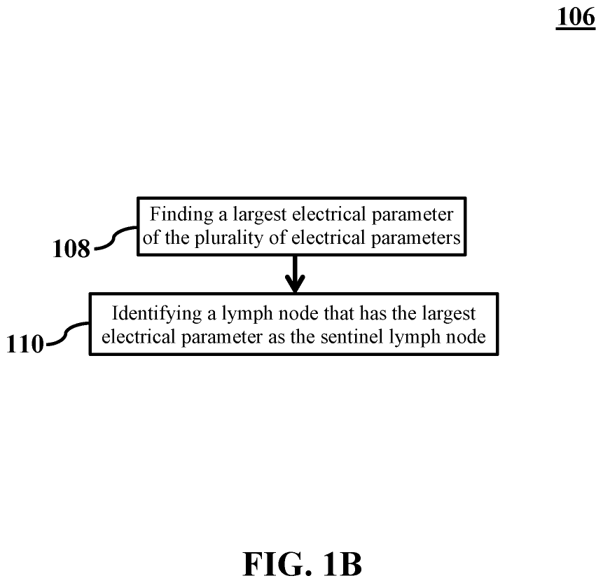

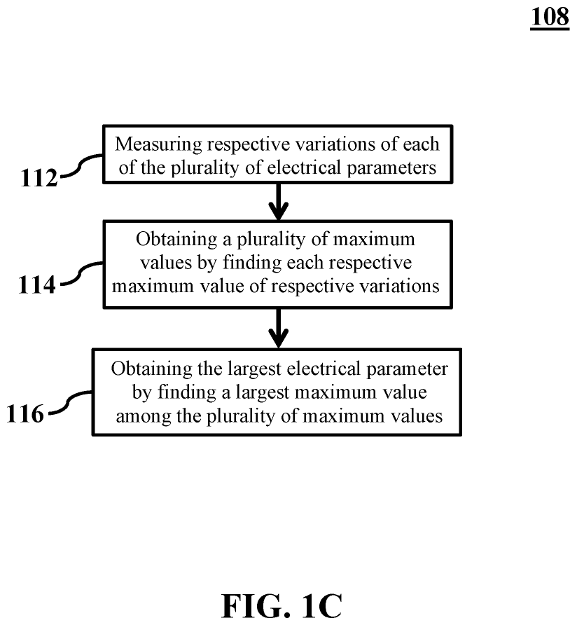

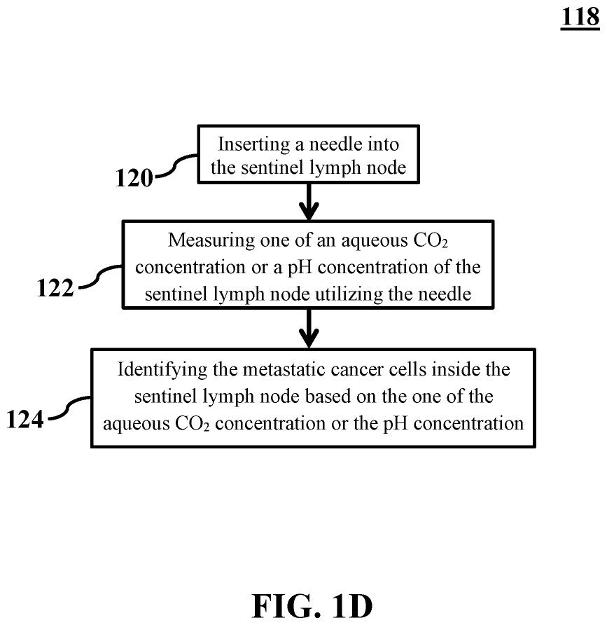

FIG. 1A shows a flowchart of a method for detecting sentinel lymph nodes during surgery, consistent with one or more exemplary embodiments of the present disclosure. An exemplary method 100 may include injecting a crystalloid solution into a region associated with a tumor in a patient's body (step 102), measuring a plurality of electrical parameters utilizing a sensor (step 104), and detecting a sentinel lymph node among a plurality of lymph nodes around the tumor based on the plurality of electrical parameters (step 106). In an exemplary embodiment, each respective lymph node of the plurality of lymph nodes may have a respective electrical parameter of the plurality of electrical parameters. Exemplary plurality of lymph nodes may be associated with the tumor and may include lymph nodes in the patient's body that may be candidates for being a sentinel lymph node. An exemplary sentinel lymph node may be located at a shortest distance from the tumor among the plurality of lymph nodes.

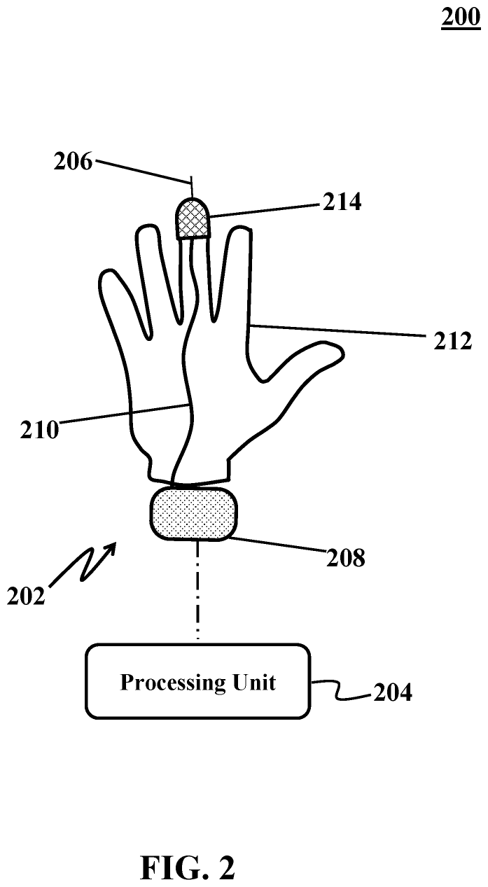

FIG. 2 shows a schematic of a system for detecting sentinel lymph nodes during surgery, consistent with one or more exemplary embodiments of the present disclosure. In an exemplary embodiment, different steps of method 100 may be implemented utilizing an exemplary system 200. In an exemplary embodiment, system 200 may include a sensor 202 and a processing unit 204. In an exemplary embodiment, sensor 202 may include a probe 206, a signal transmission unit 208, and a detachable cable 210.

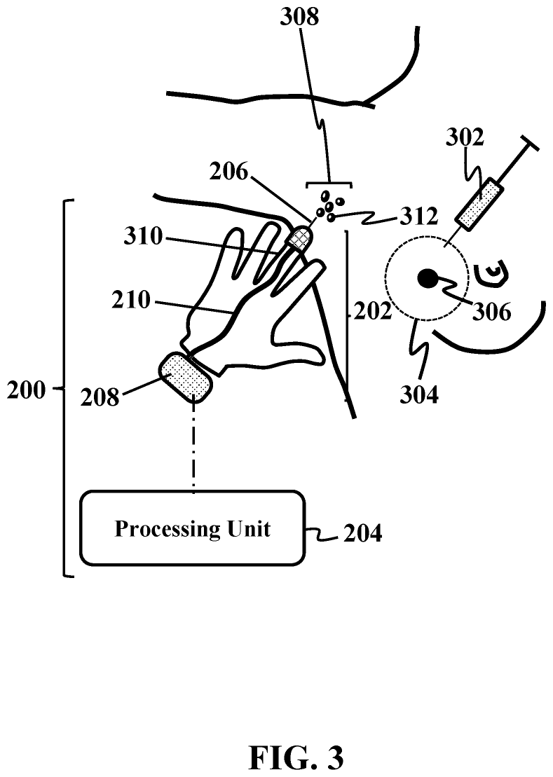

FIG. 3 shows a schematic of utilizing a system for detecting sentinel lymph nodes during surgery, consistent with one or more exemplary embodiments of the present disclosure. Referring to FIGS. 1A and 3, in an exemplary embodiment, step 102 may include injecting a crystalloid solution 302 into a region 304. An exemplary crystalloid solution may refer to an isotonic plasma volume expander that may contain an electrolyte. In an exemplary embodiment, crystalloid solution 302 may increase circulatory volume without altering chemical balance in vascular spaces due to isotonic properties of crystalloid solution 302. In an exemplary embodiment, injecting crystalloid solution 302 may include injecting one of a normal saline, a dextrose solution, or a Ringer's solution into region 304. In an exemplary embodiment, region 304 may include a vicinity surrounding a tumor 306. In an exemplary embodiment, a size of region 304 may be determined such that a majority of crystalloid solution 302 may be absorbed by tumor 306 to be eventually discharged into neighboring lymph nodes. Therefore, no exemplary lymph node may be included in region 304. In an exemplary embodiment, region 304 may be twice as large as tumor 306. In an exemplary embodiment, crystalloid solution 302 may be injected anywhere in region 304.

In an exemplary embodiment, step 104 may include measuring a plurality of electrical parameters utilizing sensor 202. In an exemplary embodiment, each of the plurality of electrical parameters may be measured by probe 206 that may be inserted into a respective lymph node of a plurality of lymph nodes 308. In an exemplary embodiment, plurality of lymph nodes 308 may include lymph nodes that may be candidates for being a sentinel lymph node for tumor 306, i.e., having a shortest distance from tumor 306. Therefore, in an exemplary embodiment, it may be known prior to executing method 100 that plurality of lymph nodes 308 may include a nearest lymph node to tumor 306 among lymph nodes in the patient's body. However, in an exemplary embodiment, a closest lymph node to tumor 306 among plurality of lymph nodes 308 may be unknown and method 100 may be implemented to find the closest lymph node. For each exemplary lymph node of plurality of lymph nodes 308 a separate parameter may be measured. In an exemplary embodiment, each of the plurality of electrical parameters may be transmitted from probe 206 to signal transmission unit 208 via detachable cable 210 after being measured by probe 206. Exemplary plurality of electrical parameters may be sent to processing unit 204 from signal transmission unit 208 for further processing through remaining steps of method 100.

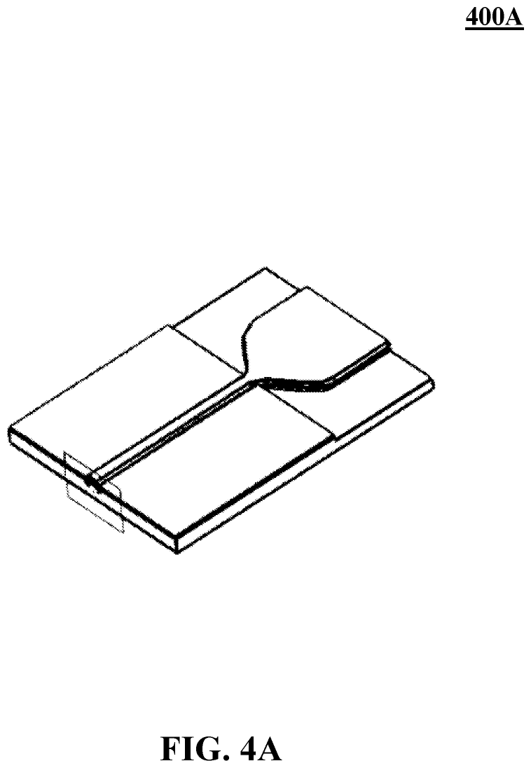

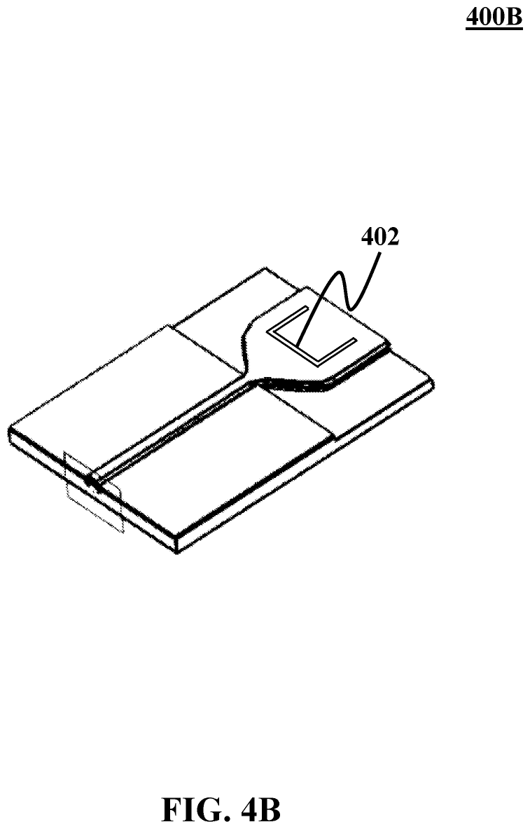

In an exemplary embodiment, sensor may include a flexible gigahertz antenna. FIG. 4A shows a schematic of a flexible coplanar waveguide (CPW) antenna, consistent with one or more exemplary embodiments of the present disclosure. An exemplary flexible CPW antenna 400A may include a microstrip antenna that may be configured to operate at a specific frequency (that is a single-frequency).

FIG. 4B shows a schematic of a flexible CPW antenna with a slot, consistent with one or more exemplary embodiments of the present disclosure. An exemplary slot 402 may be implemented on an exemplary flexible CPW antenna 400B to increase the bandwidth of flexible CPW antenna 400B. In an exemplary embodiment, corners of flexible CPW antenna 400B may be bent to improve a radiation pattern of flexible CPW antenna 400B.

Referring again to FIGS. 2 and 3, in an exemplary embodiment, sensor 202 may further include a glove 212. An exemplary mounting member 214 may be placed on one of a fingers of glove 212. In an exemplary embodiment, mounting member 214 may include a flexible cap similar to a thimble that may be worn on the user's finger. An exemplary flexible cap may be made of a flexible material such as rubber. In an exemplary embodiment, probe 206 may be attached onto mounting member 214. As a result, an exemplary operator may easily use system 200 by wearing glove 212 and may insert probe 206 into each of plurality of lymph nodes 308 by moving a respective finger on which mounting member 214 is placed (for example, finger 310) to each respective lymph node.

In an exemplary embodiment, probe 206 and mounting member 214 may be single serving and after each insertion of probe 206 in a lymph node, probe 206 and mounting member 214 may be disconnected from sensor 202 to be replaced with new ones to test other lymph nodes to avoid unwanted spread of cancer cells to healthy lymph nodes.

In an exemplary embodiment, step 106 may include detecting a sentinel lymph node during surgery. An exemplary sentinel lymph node 312 may refer to a lymph node that may be located at a shortest distance from tumor 306 among plurality of lymph nodes 308. Therefore, in an exemplary embodiment, sentinel lymph node 312 may absorb a larger portion of injected crystalloid solution 302 than other lymph nodes of plurality of lymph nodes 308. As a result, in an exemplary embodiment, sentinel lymph node 312 may be affected more by crystalloid solution 302 than other lymph nodes. Since, in an exemplary embodiment, crystalloid solution 302 may include an electrolyte which may be electrically conductive, an impact of adding crystalloid solution 302 to each of plurality of lymph nodes 308 may be observed by measuring electrical properties of each respective lymph node. Therefore, in an exemplary embodiment, sentinel lymph node 312 may be recognized from other lymph nodes based on the measured electrical properties.

For further detail with respect to step 106, FIG. 1B shows a flowchart for detecting a sentinel lymph node, consistent with one or more exemplary embodiments of the present disclosure. In an exemplary embodiment, detecting sentinel lymph node 312 may include finding a largest electrical parameter of the plurality of electrical parameters (step 108) and identifying a lymph node of plurality of lymph nodes 308 that has the largest electrical parameter as sentinel lymph node 312 (step 110). An exemplary largest electrical parameter may include a highest magnitude among the plurality of electrical parameters.

In further detail with regards to step 108, FIG. 1C shows a flowchart for finding a largest electrical parameter, consistent with one or more exemplary embodiments of the present disclosure. In an exemplary embodiment, finding the largest electrical parameter in step 108 may include measuring respective variations of each of the plurality of electrical parameters in a frequency domain utilizing sensor 202 (step 112), obtaining a plurality of maximum values by finding each respective maximum value of respective variations of each of the plurality of electrical parameters in a predetermined frequency range of the frequency domain (step 114), and obtaining the largest electrical parameter by finding a largest maximum value among the plurality of maximum values (step 116).

For further detail regarding step 112, in an exemplary embodiment, measuring respective variations of each of the plurality of electrical parameters may include measuring respective variations of one of a plurality of scattering parameters (or S-parameters), a plurality of electrical conductivities, or a plurality of relative permittivities in the frequency domain for each respective lymph node of plurality of lymph nodes 308. In an exemplary embodiment, a flexible gigahertz antenna, such as any of flexible CPW antennas 400A and 400B may be utilized for measuring variations of each of the plurality of electrical parameters by irradiating gigahertz electromagnetic waves and examining changes in each of the plurality of electrical parameters.

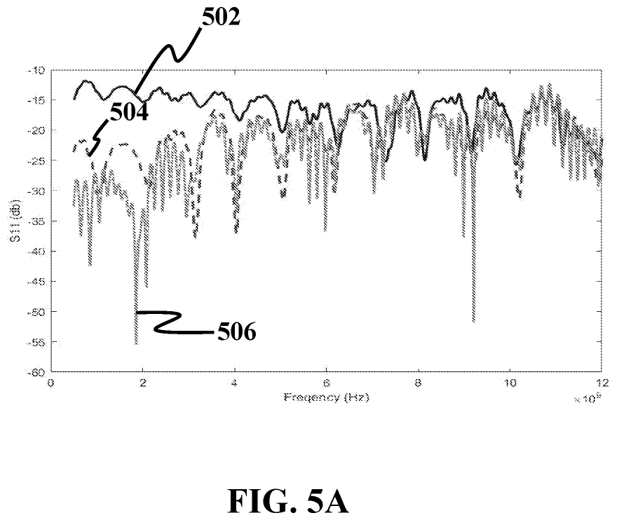

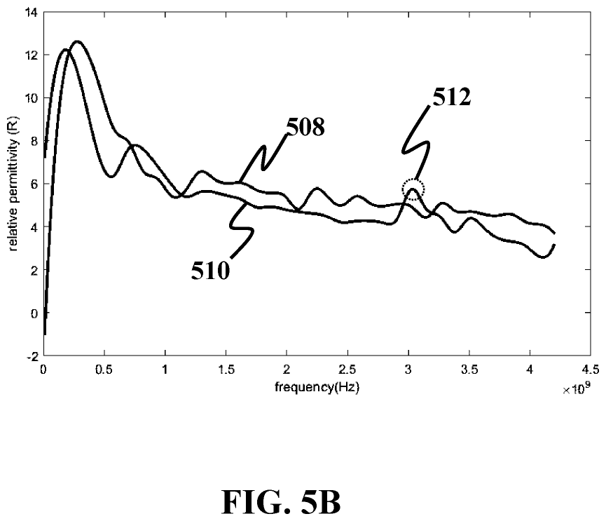

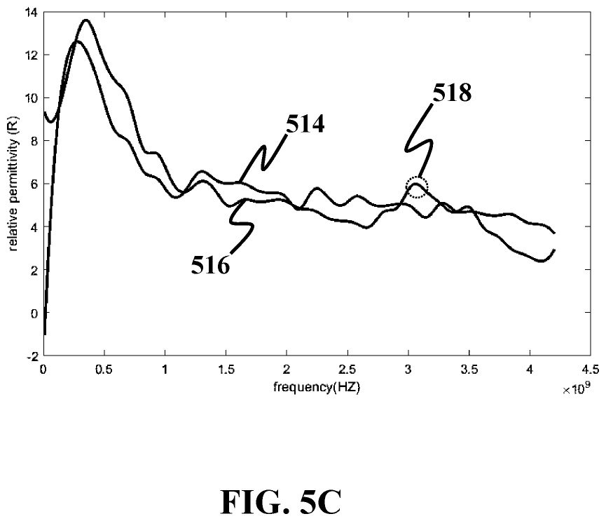

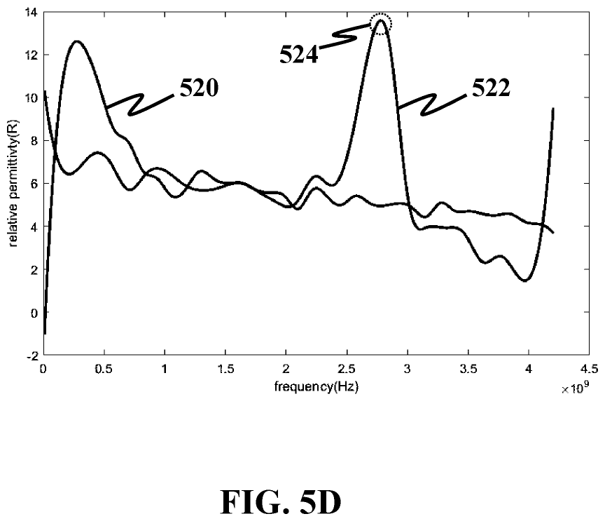

An exemplary S-parameter (i.e., an element of a scattering matrix or S-matrix) may describe the electrical behavior of linear electrical networks when being stimulated by electrical signals. FIG. 5A shows measured variations of an S-parameter in a lymph node, consistent with one or more exemplary embodiments of the present disclosure. An exemplary curve 502 illustrates variations of an S₁₁ parameter (i.e., a first element of an S-matrix) of an exemplary lymph node before injecting crystalloid solution 302. An exemplary curve 504 illustrates S₁₁ variations of an exemplary lymph nodes after crystalloid solution 302 is injected to region 304. An exemplary curve 506 illustrates S₁₁ variations about two minutes after injecting crystalloid solution 302. It may be observed in curves 504 and 506 that injecting crystalloid solution 302 may cause changes in S₁₁ variations at different frequencies, for example, in a frequency range of about 0.5 to 6 GHz.

Similarly, in an exemplary embodiment, injecting crystalloid solution 302 may cause changes in electrical conductivity (i.e., ability to conduct electric current) and relative permittivity (i.e., electric polarizability with respect to vacuum) of lymph nodes. In an exemplary embodiment, finding each respective maximum value of respective variations in step 114 may include finding each respective maximum value of respective variations of each of the plurality of relative permittivities for each respective lymph node of plurality of lymph nodes 308 in a frequency range of about 2 GHz to about 4 GHz in the frequency domain.

FIG. 5B shows measured variations of a relative permittivity in an axillary lymph node before and after injecting a dextrose solution, consistent with one or more exemplary embodiments of the present disclosure. An exemplary curve 508 illustrates relative permittivity variations of an exemplary axillary lymph node before injecting dextrose to region 304. An exemplary curve 510 illustrates relative permittivity variations of the axillary lymph node about five minutes after dextrose is injected. In an exemplary embodiment, curve 510 may include a local maximum value 512 in a frequency range of 2 to 4 GHz (at about 3 GHz).

FIG. 5C shows measured variations of a relative permittivity in an axillary lymph node before and after injecting a Ringer's solution, consistent with one or more exemplary embodiments of the present disclosure. An exemplary curve 514 illustrates relative permittivity variations of an exemplary axillary lymph node before injecting a Ringer's solution to region 304. An exemplary curve 516 illustrates relative permittivity variations of the axillary lymph node after the Ringer's solution is injected. In an exemplary embodiment, curve 516 may include a local maximum value 518 in a frequency range of 2 to 4 GHz (at about 3 GHz).

FIG. 5D shows measured variations of a relative permittivity in an axillary lymph node before and after injecting a normal saline solution, consistent with one or more exemplary embodiments of the present disclosure. An exemplary curve 520 illustrates relative permittivity variations of an exemplary axillary lymph node before injecting normal saline to region 304. An exemplary curve 522 illustrates relative permittivity variations of the axillary lymph node after normal saline is injected. In an exemplary embodiment, curve 522 may include a maximum value 524 in a frequency range of 2 to 4 GHz (at about 2.8 GHz).

In further detail with respect to step 116, in an exemplary embodiment, each of the plurality of maximum values (for example, maximum value 524) may correspond to a respective lymph node of plurality of lymph nodes 308. In an exemplary embodiment, finding the largest maximum value among the plurality of maximum values in step 116 may include finding a maximum value that is larger than the rest of the plurality of maximum values.

Referring again to FIGS. 1B and 3, in an exemplary embodiment, step 110 may include identifying a lymph node of plurality of lymph nodes 308 as sentinel lymph node 312 if said lymph node may have the largest maximum value, i.e., the largest maximum value is obtained from variations of an electrical parameter measured from said lymph node.

Referring again to FIG. 1A, in an exemplary embodiment, method may further include detecting metastatic cancer cells inside sentinel lymph node 312 by tracking FAO of metastatic cancer cells within sentinel lymph node 312 (step 118). In an exemplary embodiment, metastatic tumor cells within a lymph node may undergo a metabolic shift toward FAO and may produce CO₂ and water within the lymph node. Therefore, in an exemplary embodiment tracking FAO of metastatic cancer cells within sentinel lymph node 312 may be carried out by measuring either concentration of aqueous CO₂ or pH of sentinel lymph node 312.

FIG. 1D shows a flowchart for detecting metastatic cancer cells inside a lymph node, consistent with one or more exemplary embodiments of the present disclosure. In an exemplary embodiment, step 118 may include inserting a needle of sensor 202 into sentinel lymph node 312 (step 120), measuring one of an aqueous CO₂ concentration or a pH concentration of sentinel lymph node 312 utilizing the needle (step 122), and identifying the metastatic cancer cells inside sentinel lymph node 312 based on the one of the aqueous CO₂ concentration or the pH concentration (step 124).

For further detail with regards to step 120, an exemplary the needle may include one of a miniaturized CO₂ sensor needle or a miniaturized pH sensor needle. In an exemplary embodiment, a CO₂ sensor may refer to a sensor that may be configured to measure aqueous CO₂ concentration in a lymph node. An exemplary pH sensor may refer to a sensor that may be configured to measure pH concentration in a lymph node. Referring again to FIGS. 2 and 3, in an exemplary embodiment, inserting the needle into sentinel lymph node 312 may include replacing probe 206 with the needle in sensor 202 and utilizing sensor 202 as shown in FIG. 3 and described above.

In further detail regarding step 122, an exemplary miniaturized CO₂ sensor needle may be utilized to measure aqueous CO₂ concentration in sentinel lymph node 312. In order to measure pH concentration of sentinel lymph node 312, an exemplary miniaturized pH sensor needle may be utilized in sensor 202. In an exemplary embodiment, sensor 202 may measure either of aqueous CO₂ concentration or pH concentration when the needle is inserted into sentinel lymph node 312. Exemplary measured data may be sent to processing unit 204 for further processing after measurement is done.

In further detail regarding step 124, in an exemplary embodiment, processing unit 204 may compare the measured data with a predetermined cancer threshold. An exemplary cancer threshold may be obtained based on measurements from known cancerous lymph nodes prior to the surgery. In an exemplary embodiment, aqueous CO₂ concentration and pH concentration may be higher in cancerous lymph nodes than in normal lymph nodes. Therefore, an exemplary predetermined cancer threshold may be set between a lower limit and an upper limit. An exemplary upper limit may be set to a minimum aqueous CO₂ concentration of a number of aqueous CO₂ concentrations or a minimum pH concentration of a number of pH concentrations that may be measured from a number of known cancerous lymph nodes. An exemplary lower limit may be set to a maximum aqueous CO₂ concentration of a number of aqueous CO₂ concentrations or a maximum pH concentration of a number of pH concentrations that may be measured from a number of known normal lymph nodes. An exemplary predetermined cancer threshold may be utilized to distinguish a cancerous lymph node from a normal lymph node. In an exemplary embodiment, processing unit 204 may identify sentinel lymph node 312 as a cancerous tissue if the measured data is larger than the predetermined cancer threshold.

In an exemplary embodiment, a cancerous lymph node regardless of being a sentinel lymph node or not may be detected by steps 122-124. In other words, an exemplary cancerous lymph node, sentinel or not, may be detected by inserting a miniaturized CO₂ sensor needle or a miniaturized pH sensor needle into the lymph node and measuring either concentration of aqueous CO₂ or pH of the lymph node, and determining if the lymph node is cancerous or not by comparing CO₂ or pH levels of the lymph node with those of a healthy lymph node.

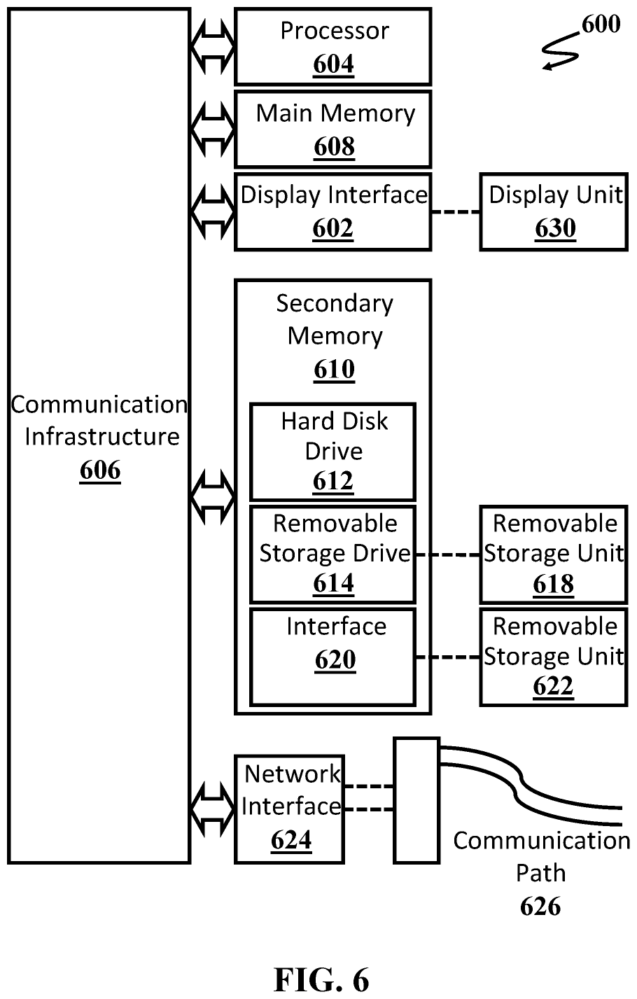

FIG. 6 shows an example computer system 600 in which an embodiment of the present invention, or portions thereof, may be implemented as computer-readable code, consistent with exemplary embodiments of the present disclosure. For example, method 100 may be implemented in computer system 600 using hardware, software, firmware, tangible computer readable media having instructions stored thereon, or a combination thereof and may be implemented in one or more computer systems or other processing systems. In an exemplary embodiment, system 600 may be analogous to processing unit 204. Hardware, software, or any combination of such may embody any of the modules and components in FIGS. 1A-3.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 600. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 604 may be a special purpose or a general-purpose processor device. As will be appreciated by persons skilled in the relevant art, processor device 604 may also be a single processor in a multi-core/multiprocessor system, such system operating alone, or in a cluster of computing devices operating in a cluster or server farm. Processor device 604 may be connected to a communication infrastructure 606, for example, a bus, message queue, network, or multi-core message-passing scheme.

In an exemplary embodiment, computer system 600 may include a display interface 602, for example a video connector, to transfer data to a display unit 630, for example, a monitor. Computer system 600 may also include a main memory 608, for example, random access memory (RAM), and may also include a secondary memory 610. Secondary memory 610 may include, for example, a hard disk drive 612, and a removable storage drive 614. Removable storage drive 614 may include a floppy disk drive, a magnetic tape drive, an optical disk drive, a flash memory, or the like. Removable storage drive 614 may read from and/or write to a removable storage unit 618 in a well-known manner. Removable storage unit 618 may include a floppy disk, a magnetic tape, an optical disk, etc., which may be read by and written to by removable storage drive 614. As will be appreciated by persons skilled in the relevant art, removable storage unit 618 may include a computer usable storage medium having stored therein computer software and/or data.

In alternative implementations, secondary memory 610 may include other similar means for allowing computer programs or other instructions to be loaded into computer system 600. Such means may include, for example, a removable storage unit 622 and an interface 620. Examples of such means may include a program cartridge and cartridge interface (such as that found in video game devices), a removable memory chip (such as an EPROM, or PROM) and associated socket, and other removable storage units 622 and interfaces 620 which allow software and data to be transferred from removable storage unit 622 to computer system 600.

Computer system 600 may also include a communications interface 624. Communications interface 624 allows software and data to be transferred between computer system 600 and external devices. Communications interface 624 may include a modem, a network interface (such as an Ethernet card), a communications port, a PCMCIA slot and card, or the like. Software and data transferred via communications interface 624 may be in the form of signals, which may be electronic, electromagnetic, optical, or other signals capable of being received by communications interface 624. These signals may be provided to communications interface 624 via a communications path 626. Communications path 626 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 618, removable storage unit 622, and a hard disk installed in hard disk drive 612. Computer program medium and computer usable medium may also refer to memories, such as main memory 608 and secondary memory 610, which may be memory semiconductors (e.g. DRAMs, etc.).

Computer programs (also called computer control logic) are stored in main memory 608 and/or secondary memory 610. Computer programs may also be received via communications interface 624. Such computer programs, when executed, enable computer system 600 to implement different embodiments of the present disclosure as discussed herein. In particular, the computer programs, when executed, enable processor device 604 to implement the processes of the present disclosure, such as the operations in method 100 illustrated by flowcharts of FIG. 1A-FIG. 1D discussed above. Accordingly, such computer programs represent controllers of computer system 600. Where an exemplary embodiment of method 100 is implemented using software, the software may be stored in a computer program product and loaded into computer system 600 using removable storage drive 614, interface 620, and hard disk drive 612, or communications interface 624.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for detecting sentinel lymph nodes during surgery, the method comprising:
injecting a normal saline into a tumor in a patient's body;
measuring, utilizing a flexible gigahertz antenna, respective variations of relative permittivities of each respective lymph node of a plurality of lymph nodes associated with the tumor in a frequency domain by inserting a probe of the flexible gigahertz antenna into each of the plurality of lymph nodes; and
detecting, utilizing one or more processors, a sentinel lymph node among the plurality of lymph nodes, the sentinel lymph node located at a shortest distance from the tumor among the plurality of lymph nodes, detecting the sentinel lymph node comprising:
obtaining a plurality of maximum values by finding each respective maximum value of respective variations of each respective relative permittivity of each respective lymph node of the plurality of lymph nodes in a predetermined frequency range of the frequency domain;
finding a largest maximum value among the plurality of maximum values; and
identifying a lymph node of the plurality of lymph nodes that comprises the largest maximum value as the sentinel lymph node.

2. The method of claim 1, wherein obtaining the plurality of maximum values in the predetermined frequency range comprises finding each respective maximum value of respective variations of each respective relative permittivity of each respective lymph node of the plurality of lymph nodes in in a frequency range of 2 GHz to 4 GHz in the frequency domain.

3. A method for detecting sentinel lymph nodes during surgery, the method comprising:
injecting a crystalloid solution into a region associated with a tumor in a patient's body;
measuring, utilizing a sensor, a plurality of electrical parameters by measuring each respective electrical parameter of a respective lymph node of a plurality of lymph nodes, the plurality of lymph nodes associated with the tumor; and
detecting, utilizing one or more processors, a sentinel lymph node among the plurality of lymph nodes based on the plurality of electrical parameters, the sentinel lymph node located at a shortest distance from the tumor among the plurality of lymph nodes.

4. The method of claim 3, wherein detecting the sentinel lymph node comprises:
finding a largest electrical parameter of the plurality of electrical parameters, the largest electrical parameter comprising a highest magnitude among the plurality of electrical parameters; and
identifying a lymph node of the plurality of lymph nodes that comprises the largest electrical parameter as the sentinel lymph node.

5. The method of claim 4, wherein finding the largest electrical parameter comprises:
measuring, utilizing the sensor, respective variations of each of the plurality of electrical parameters in a frequency domain;
obtaining a plurality of maximum values by finding each respective maximum value of respective variations of each of the plurality of electrical parameters in a predetermined frequency range of the frequency domain; and
obtaining the largest electrical parameter by finding a largest maximum value among the plurality of maximum values.

6. The method of claim 5, wherein measuring respective variations of each of the plurality of electrical parameters comprises measuring respective variations of one of a plurality of scattering parameters, a plurality of electrical conductivities, or a plurality of relative permittivities in the frequency domain for each respective lymph node of the plurality of lymph nodes.

7. The method of claim 6, wherein injecting the crystalloid solution comprises injecting one of a normal saline, a dextrose solution, or a Ringer's solution into the region.

8. The method of claim 7, wherein finding each respective maximum value of respective variations of each of the plurality of electrical parameters in the predetermined frequency range comprises finding each respective maximum value of respective variations of each of the plurality of relative permittivities for each respective lymph node of the plurality of lymph nodes in a frequency range of 2 GHz to 4 GHz in the frequency domain.

9. The method of claim 3, wherein utilizing the sensor comprises:
inserting a probe of a flexible gigahertz antenna into each of the plurality of lymph nodes, the flexible gigahertz antenna comprising a flexible coplanar waveguide (CPW) antenna;
measuring each of the plurality of electrical parameters by the probe;
transmitting each of the plurality of electrical parameters from the probe to a signal transmission unit via a detachable cable; and
transmitting the plurality of electrical parameters to the one or more processors from the signal transmission unit.

10. The method of claim 9, wherein inserting the probe into each of the plurality of lymph nodes comprises:
wearing a glove by an operator of the sensor;
placing a mounting member on one of a fingers of the glove; and
attaching the probe onto the mounting member.

11. The method of claim 3, wherein injecting the crystalloid solution into the region comprises injecting the crystalloid solution into the tumor.

12. The method of claim 3, further comprising detecting metastatic cancer cells inside the sentinel lymph node by:
inserting a needle of the sensor into the sentinel lymph node, the needle comprising one of a miniaturized carbon dioxide (CO₂) sensor needle or a miniaturized pH sensor needle;
measuring, utilizing the needle, one of an aqueous CO₂ concentration or a pH concentration of the sentinel lymph node; and
identifying the metastatic cancer cells inside the sentinel lymph node based on the one of the aqueous CO₂ concentration or the pH concentration.

13. A system for detecting sentinel lymph nodes during surgery, the system comprising:
a sensor configured to measure a plurality of electrical parameters by measuring each respective electrical parameter of a respective lymph node of a plurality of lymph nodes, the plurality of lymph nodes located in a region associated with a tumor in a patient's body, the region comprising a crystalloid solution injected into the tumor;
a memory having processor-readable instructions stored therein; and
one or more processors configured to access the memory and execute the processor-readable instructions, which, when executed by the processor configures the one or more processors to perform a method, the method comprising:
detecting a sentinel lymph node among the plurality of lymph nodes based on the plurality of electrical parameters, the sentinel lymph node located at a shortest distance from the tumor among the plurality of lymph nodes.

14. The system of claim 13, wherein detecting the sentinel lymph node comprises:
measuring, utilizing the sensor, respective variations of each of the plurality of electrical parameters in a frequency domain;
finding a largest electrical parameter of the plurality of electrical parameters, the largest electrical parameter comprising a highest magnitude in the frequency domain among the plurality of electrical parameters; and
identifying a lymph node of the plurality of lymph nodes that comprises the largest electrical parameter as the sentinel lymph node.

15. The system of claim 14, wherein finding the largest electrical parameter comprises:
obtaining a plurality of maximum values by finding each respective maximum value of respective variations of each of the plurality of electrical parameters in a predetermined frequency range of the frequency domain; and
obtaining the largest electrical parameter by finding a largest maximum value among the plurality of maximum values.

16. The system of claim 15, wherein measuring respective variations of each of the plurality of electrical parameters comprises measuring respective variations of one of a plurality of scattering parameters, a plurality of electrical conductivities, or a plurality of relative permittivities in the frequency domain for each respective lymph node of the plurality of lymph nodes.

17. The system of claim 16, wherein finding each respective maximum value of respective variations of each of the plurality of electrical parameters in the predetermined frequency range comprises finding each respective maximum value of respective variations of each of the plurality of relative permittivities for each respective lymph node of the plurality of lymph nodes in a frequency range of 2 GHz to 4 GHz in the frequency domain.

18. The system of claim 13, wherein the sensor comprises:
a glove configured to be worn by an operator of the sensor;
a mounting member configured to be placed on one of a fingers of the glove;
a flexible coplanar waveguide (CPW) antenna comprising a probe attached onto the mounting member, the probe configured to:
be inserted into each of the plurality of lymph nodes; and
measure each of the plurality of electrical parameters;
a signal transmission unit configured to transmit the plurality of electrical parameters to the one or more processors; and
a detachable cable configured to transmit each of the plurality of electrical parameters from the probe to the signal transmission unit.

19. The system of claim 18, wherein the sensor further comprises a needle comprising one of a miniaturized carbon dioxide (CO₂) sensor needle or a miniaturized pH sensor needle, the needle configured to measure one of an aqueous CO₂ concentration or a pH concentration of the sentinel lymph node.

20. The system of claim 19, wherein the method further comprises detecting metastatic cancer cells inside the sentinel lymph node based on the one of the aqueous CO₂ concentration or the pH concentration.
